(19) United States
(12) Patent Application Publication
(10) Pub. No.: US 2026/0004913 A1
(43) Pub. Date: Jan. 1, 2026

(54) CONTRASTIVE MULTI-OMICS ASSOCIATION LEARNING FOR COMPLEX DISEASES

(71) Applicants: International Business Machines Corporation, Armonk, NY (US); Rensselaer Polytechnic Institute, Troy, NY (US)

(72) Inventors: ARITRA BOSE, White Plains, NY (US); Diego Machado Reyes, Atlixco (MX); Myson Burch, West Harrison, NY (US); Laxmi Parida, Mohegan Lake, NY (US)

(21) Appl. No.: 18/756,606

(22) Filed: Jun. 27, 2024

(51) Int. Cl. G16H 20/40; G16H 50/20

(52) U.S. Cl. CPC G16H 20/40; G16H 50/20

(57) ABSTRACT

A plurality of data pairs are created by matching an element from a first modality with an element from a second modality. Each element from the first modality and each element from the second modality are tokenized to obtain first modality tokens and second modality tokens. A model is trained based on the plurality of data pairs, the training comprising learning a first embedding from the first modality tokens via a first attention-based encoder for the first modality and a second embedding from the second modality tokens via a second attention-based encoder for the second modality, calculating a cosine similarity between the first embedding and the second embedding for each data pair and computing a loss between predicted items and ground truth based on the cosine similarity. The predicted items with a minimal loss are validated to obtain at least one candidate therapeutic.

### BACKGROUND

The present invention relates generally to the electrical, electronic and computer arts and, more particularly, to machine learning and medical diagnostic and therapeutic technology.

Complex diseases are usually a result of complex interactions between a combined effect of multiple genes, commonly known as the polygenic effect, and multiple phenotypes or traits often induced by their symptoms. Neurological diseases, such as Alzheimer's disease, Parkinson's disease, bipolar disorder and the like, are often captured by different modalities of health care data, such as imaging, genetics, blood biochemistry measures and the like. Hence, to understand these complex diseases, it is pertinent to better understand how various modalities are associated with each other, and how some modalities impact the disease. As these multiple modalities span across genomics, phenomics, transcriptomics, radiomics, proteomics, and the like, the multiple modalities are known as multi-omics data in healthcare and life sciences parlance.

### BRIEF SUMMARY

Principles of the invention provide systems and techniques for contrastive multi-omics association learning for complex diseases. In one aspect, an exemplary method includes the operations of creating a plurality of data pairs by matching an element from a first modality with an element from a second modality; tokenizing each element from the first modality and each element from the second modality to obtain first modality tokens and second modality tokens; training a model based on the plurality of data pairs, the training comprising learning a first embedding from the first modality tokens via a first attention-based encoder for the first modality and a second embedding from the second modality tokens via a second attention-based encoder for the second modality, calculating a cosine similarity between the first embedding and the second embedding for each data pair and computing a loss between predicted items and ground truth based on the cosine similarity; and validating the predicted items with a minimal loss to obtain at least one candidate therapeutic.

In one aspect, a computer program product comprises one or more tangible computer-readable storage media and program instructions stored on at least one of the one or more tangible computer-readable storage media, the program instructions executable by a processor, the program instructions comprising creating a plurality of data pairs by matching an element from a first modality with an element from a second modality; tokenizing each element from the first modality and each element from the second modality to obtain first modality tokens and second modality tokens; training a model based on the plurality of data pairs, the training comprising learning a first embedding from the first modality tokens via a first attention-based encoder for the first modality and a second embedding from the second modality tokens via a second attention-based encoder for the second modality, calculating a cosine similarity between the first embedding and the second embedding for each data pair and computing a loss between predicted items and ground truth based on the cosine similarity; and validating the predicted items with a minimal loss to obtain at least one candidate therapeutic.

In one aspect, a system comprises a memory and at least one processor, coupled to the memory, and operative to perform operations comprising creating a plurality of data pairs by matching an element from a first modality with an element from a second modality; tokenizing each element from the first modality and each element from the second modality to obtain first modality tokens and second modality tokens; training a model based on the plurality of data pairs, the training comprising learning a first embedding from the first modality tokens via a first attention-based encoder for the first modality and a second embedding from the second modality tokens via a second attention-based encoder for the second modality, calculating a cosine similarity between the first embedding and the second embedding for each data pair and computing a loss between predicted items and ground truth based on the cosine similarity; and validating the predicted items with a minimal loss to obtain at least one candidate therapeutic.

These and other features and advantages will become apparent from the following detailed description of illustrative embodiments thereof, which is to be read in connection with the accompanying drawings.

### BRIEF DESCRIPTION OF THE DRAWINGS

The following drawings are presented by way of example only and without limitation, wherein like reference numerals (when used) indicate corresponding elements throughout the several views, and wherein:

FIG. 1 illustrates a workflow for performing contrastive learning for multi-omics association learning, in accordance with an example embodiment;

FIG. 2 illustrates a flowchart for performing contrastive learning for multi-omics association learning, in accordance with an example embodiment;

FIG. 3 is a table illustrating experimental results, in accordance with an example embodiment;

FIG. 4 illustrates tokenization of genome information for a given single nucleotide polymorphisms (SNP), in accordance with an example embodiment;

FIG. 5 illustrates tokenization of brain image information for a given imaging derived phenotype, in accordance with an example embodiment;

FIG. 6 illustrates an example of pair-making between imaging-derived phenotypes (IDPs) and SNPs, in accordance with an example embodiment; and

FIG. 7 depicts a computing environment according to an embodiment of the present invention.

It is to be appreciated that elements in the figures are illustrated for simplicity and clarity. Common but well-understood elements that may be useful or necessary in a commercially feasible embodiment may not be shown in order to facilitate a less hindered view of the illustrated embodiments.

### DETAILED DESCRIPTION

Principles of inventions described herein will be in the context of illustrative embodiments. Moreover, it will become apparent to those skilled in the art given the teachings herein that numerous modifications can be made to the embodiments shown that are within the scope of the claims. That is, no limitations with respect to the embodiments shown and described herein are intended or should be inferred.

The treatment of complex diseases typically requires a comprehensive understanding of patients and their histories, from a multi-modal data set spanning across electronic medical records (EMRs) to molecular profiling, from the whole genomic or transcriptome, to proteome sequencing to imaging data from many timepoints, often referred to collectively as multi-omics. An important challenge is to parse the multi-omics data and find interpretable associations between them for accelerating the discovery of therapeutics. Discovering these associations is conventionally done by computing genome-wide association studies (GWAS) which find single genetic marker associations with the phenotypes of interest, but this can be restrictive in many ways. In one example embodiment, a contrastive learning approach to multi-omics data is disclosed for obtaining many-to-many associations between any two types of multi-omics data.

Most complex diseases are polygenic; obtaining a holistic view of relationships between multi-omics features will lead to the discovery of more informed and targeted therapeutics. The limited availability of annotated multimodal medical data is a key challenge to discovering multi-omics relationships, especially for rare and complex diseases. Pre-trained models, such as the one disclosed herein, allow for accelerated discovery of multi-omics relationships through fine-tuning on genomic datasets and large biobanks.

### Description of CONMOAL

The contrastive multi-omics association learning (CONMOAL) platform can be used to develop a contrastive loss between any two sets of data, utilize the contrastive loss to build a loss function, and directly provide:
a transfer-based multi-omics method to integrate various modalities of healthcare data including EMR, imaging and multi-omics data, such as genomics, transcriptomics, and proteomics;
interpretable multi-omics associations for a given complex disease;
a non-linear way of computing high-dimensional multi-omics association studies (with dimensions on the order of hundreds of thousands to millions for mutations, 20,000-30,000 for genes and the like); and
a method to learn many-to-many semantic relationships from different modalities.

CONMOAL has numerous use cases, including:
1) multi-omics association for therapeutic discovery;
2) a pre-trained omics data encoder for precision medicine; and
3) attention-based interpretable multi-omics association for ontology annotation.

In one or more embodiments, different modes of multi-omics and healthcare related features are considered in concert to build interpretable associations. One or more embodiments are suitable for processing any correlated data, including binary (e.g. diagnostic (Dx), prescriptive (Rx), habits/behavior, demographics and the like), diploid genotypic data, continuous variables (blood measurements, anthropometric phenotypes, environmental variables and the like) and imaging phenotypes based on real data. An exemplary modality-agnostic associative model enables the support of a wide diversity of data modalities. In example embodiments, application of the modality-agnostic associative model is extended beyond finding interpretable associations between SNPs and IDPs to any two suitable types of data where there is considered to be a relationship to be learned. In an example framework, any different modes of data are tokenized appropriately and treated similarly to SNPs/IDPs.

Generally, techniques are provided for parsing multi-omics data and finding interpretable associations between them. The interpretable associations are useful for accelerating the discovery of therapeutics. In one example embodiment, a contrast learning approach for multi-omics data is defined for obtaining many-to-many associations between any two types of multi-omics data. A key challenge in parsing multi-omics data and finding interpretable associations between them is the limited availability of annotated multi-omics data across all modalities, especially for rare and complex diseases. In one example embodiment, a pretrained model with embeddings learned from multimodal data is proposed to provide a way to obtain inference capabilities using multimodal data even with a lack of available training data. The pretrained model can be shared securely across platforms. The many-to-many multi-omics associations generated by the disclosed systems along with the pretrained models allow for accelerated discovery of multi-omics relationships through fine-tuning on genomic data sets and large biobanks.

Due to the sophisticated nature of complex diseases, finding interpretable associations between multi-omics data can be challenging using standard approaches. In one aspect, an exemplary contrastive learning approach leveraging multi-omics data provides the following:
a) the generation of many-to-many associations between any two types of multi-omics information via self-supervised learning schemes;
b) generation of learnable embeddings from tokenization of each modality and utilization of attention-based encoders to learn the connections between them; and
c) a pretrained model for many-to-many multi-omics association discovery via direct inference or fine-tuning on external data.

To evaluate exemplary embodiments, connections between different modalities of healthcare data, brain imaging and genetics for a variety of neurological and neurodevelopmental disorders were identified. One exemplary approach discovered several many-to-many associations between single nucleotide polymorphisms (SNPs) and imaging-derived phenotypes (IDPs) that are validated in the literature to have strong associations with neurological and psychiatric conditions. This illustrates the ability of one or more embodiments to unravel complex interactions within complex disorders and reveal relationships across modalities.

In one example embodiment, training is carried out on a paired set of imaging-derived phenotypes (IDPs) and single nucleotide polymorphisms (SNPs), a data set of approximately 40,000 individuals with 139 IDPs and 2500 SNPs, based on their relationship to neurological conditions such as Alzheimer's disease (AD), Parkinson's disease (PD), autism spectrum disorder (ASD), attention deficit hyperactivity disorder (ADHD), bipolar disorder (BD), mood disorder (MD), multiple sclerosis (MS), unipolar depression (UD) and the like.

In one example embodiment, the following stages are implemented:
1. Pair-making: positive pairs between IDPs and SNPs are defined as ground truth in order to perform the contrastive training. In one example embodiment, positive pairs were defined using a set of eight diseases (AD, ADHD, ASD, BD, MD, MS, PD, UD) as a proxy to establish the link between IDPs and SNPs. Three main steps are performed to achieve this:
a) establish buckets of SNPs related to each disease, and establish buckets of IDPs related to each disease;
b) expand the data (such as the variables, features and the like in the database) so that each data item becomes a combined record of SNPs and IDPs; and
c) for each disease, create data dictionaries relating, for each IDP, the SNPs that are associated with the same disease.
2. Genome tokenization: the contents of the pairs pertaining to SNPs are tokenized to learn high dimensional embeddings that capture the semantic relationships between them. The tokenization is performed using the following steps:
a) create a dictionary (vocabulary of tokens) of the mutation types (nucleotide substitutions, insertions and deletions);
b) for each patient, recode their mutation status into one of the tokens described above;
c) using the tokenized representation, select the corresponding learnable embedding from the deep learning methods, non-limiting examples of which are a PyTorch method, a TensorFlow method and the like.
3. IDP tokenization: as IDPs are more closely related to tabular data then sequence data, an alternative approach is taken for the IDP tokenization. Following piecewise embeddings from the patient data, each IDP is first binarized and embeddings are calculated for each sample based on their value relative to the rest of the samples. An n-dimensional vector is built in which all the values are ones (1) before the main bin, and all the values following the main bin are zeros (0). This is analogous to filling a glass of water; the glass is full up to a certain level.
4. Encoding: two independent single modality transformer encoders are used to learn representations of the SNPs and IDPs. The transformer encoders are implemented using, for example, layers in a machine learning workflow, a non-limiting example of which are the layers available in PyTorch 1.7.
5. Contrastive learning is applied to the encoded data through a self-supervised framework.
6. Computing loss: a loss is computed using two cross-entropy losses (one for each modality) and averaging them. The ground truth is established as the index of predictions of the model; that is, it is expected that the model will predict the corresponding IDP position for a given SNP in the position.
7. Model evaluation: accuracy and predictions are computed by adding up all the correct predictions in the given batch for IDP or SNP inputs, and then dividing the totals that are correct by two. A correct prediction is defined as, if given a certain IDP or SNP, a corresponding SNP or IDP from the bucket defined at the pair creation stage is predicted.

Using exemplary models, an accuracy of 97.3% in predicting SNP and IDP pairs was obtained using the 0.05% of associated SNPs (15 SNPs and 112 IDPs) with the neurological diseases without further hyperparameter tuning. The experimental results showed the effective performance of CONMOAL to learn multi-omics associations. The best model to test on was chosen using the model with the lowest loss on the validation set, which was the model at the third epoch. The best model used CONMOAL as trained using a learning rate of 0.0001, an Adam Optimizer and a cross entropy loss.

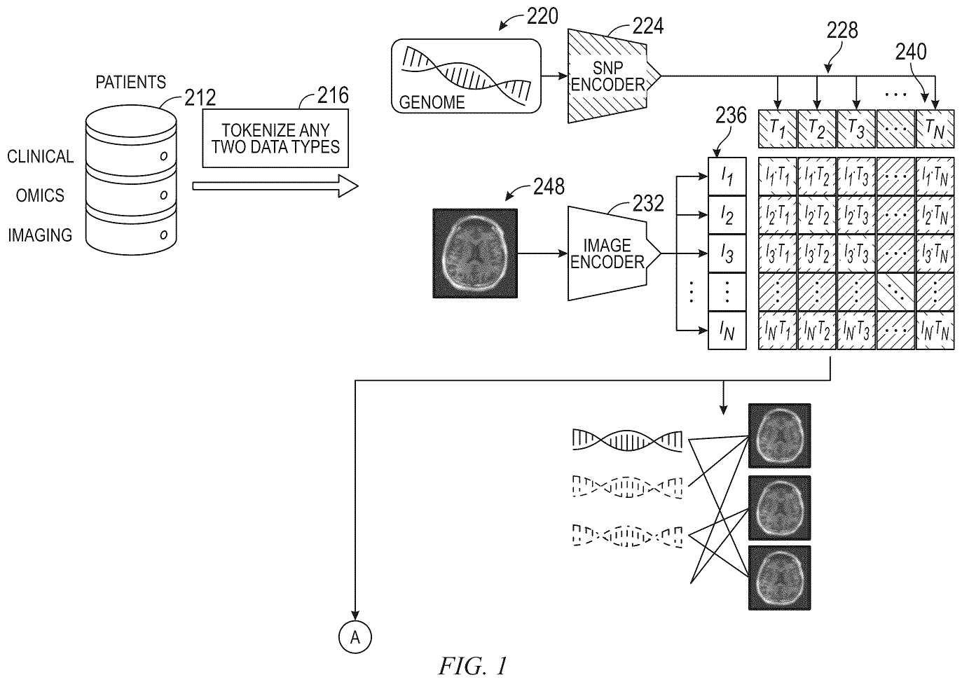

FIG. 1 illustrates a workflow for performing contrastive learning for multi-omics association learning, in accordance with an example embodiment. In one example embodiment, a database 212 contains multi-omics, including clinical information (such as clinical results), imaging, and the like for each of a plurality of patients. In one example embodiment, single nucleotide polymorphisms (SNPs: a genomic variant at a single base position in the DNA) are tokenized by tokenizer 216 and the SNPs 220 are fed into an SNP encoder 224, a single-modality attention-based encoder, to generate SNP embeddings 228. The SNP encoder 224 may be implemented with a large language model (LLM), a Generative Pre-trained Transformer (GPT) and the like.

Similarly, imaging-derived phenotypes (IDPs) are tokenized by tokenizer 216 and the IDPs 248 are fed into an image encoder 232, a single-modality attention-based encoder, to generate IDP embeddings 236. The image encoder 232 may be implemented with an LLM, a GPT and the like. A matrix 240 of data pairs is generated by matching embeddings from both modalities, such as SNP embeddings 228 and IDP embeddings 236. In essence, the data pairs of the matrix 240 relate an area of the brain with each genomic signature, and help in identifying markers. A Manhattan plot 244 of p-value vs. chromosomal location is generated using the matrix 240.

### CONMOAL Algorithm

In one example embodiment, the input to the CONMOAL platform includes multi-modal binary, categorical and continuous variables from different modalities, and the output includes a continuous variable of getting the disease.

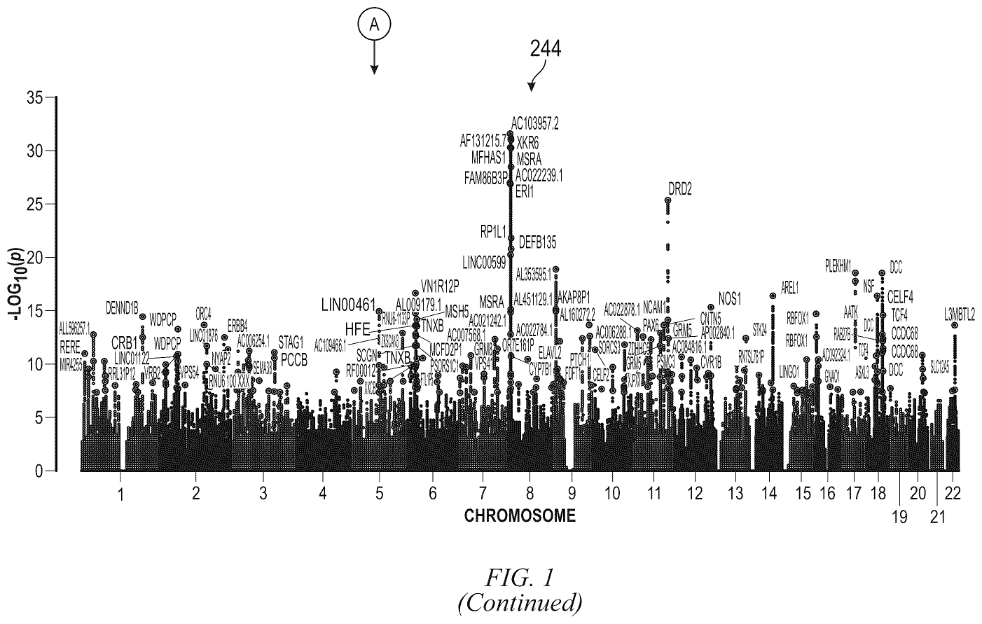

FIG. 2 illustrates a flowchart 300 for performing contrastive learning for multi-omics association learning, in accordance with an example embodiment. In one example embodiment, quality control of the input data is performed across different modalities (operation 304). For example, one or more individuals in the database 212 that are related to each other may be removed from the database 212, individuals having relevant missing information may be removed from the database 212 and the like. Data pairs are created by matching elements from both modalities (operation 308). All possible data pairs may be generated, or only the relevant data pairs may be generated and maintained, as described further below. In one example embodiment, training, validation and test splits of the data pairs (such as 70%/20%/10%) are set (operation 312).

### Training

Data from each modality is tokenized (operation 316). For sequence data, a vocabulary based on all the possible distinct elements is created. For tabular data, piecewise encodings are employed. Learnable embeddings are assigned to each token (operation 320) and the embeddings are learned through single-modality attention-based encoders 224, 232 (operation 324). For example, the single nucleotide polymorphisms are tokenized by tokenizer 216 and the tokenized SNPs 220 are fed into an SNP encoder 224 to generate the SNP embeddings 228, and the imaging-derived phenotypes (IDPs) are tokenized by tokenizer 216 and the tokenized IDPs 248 are fed into an image encoder 232 to generate the IDP embeddings 236. The various combinations of an SNP embedding 228 and an IDP embedding 236 are paired together.

The cosine similarity between the SNP embedding 228 and the IDP embedding is calculated for each data pair (operation 328). The pair relationships which are meaningful are determined by, for example, comparing the cosine similarity to a given threshold. A threshold value of 5×10⁻⁸ can be used, for example. In example embodiments, the most relevant pairs are identified with a threshold of 10⁻³⁰ to 10⁻⁴⁰. (The skilled artisan can determine the threshold heuristically depending on the domain and, in non-limiting examples or, if desired to reduce the number of pairs, can use an intermediate value.) In one example embodiment, a logistic regression technique is used generate meaningful pairs, but it is noted that such a technique does not take into consideration the context of the relationships, as described above. The matrix 240 is created using the meaningful data pairs after the meaningful data pairs are determined or the matrix 240 is created using all data pairs and the unmeaningful data pairs are then filtered out of the matrix 240 after the meaningful data pairs are determined.

The loss between the predicted item and the ground truth is computed based on cross-modality pairs and the cosine similarity of operation 328 (operation 332).

### Evaluation

The best performing epoch (model and corresponding set of parameters) is selected, where the best model is tested using the validation dataset (operation 336). The mode on a test dataset is evaluated by calculating the accuracy (operation 340). In one example test, based on an input SNP or IDP, a determination is made of whether the model retrieves a matching IDP or SNP, respectively, from the pairing defined at the beginning.

In one example embodiment, a model is trained based on the data pairs and/or the matrix 240 (operation 344). The model can then be finetuned on another training data set, such as the training data set of another user.

Candidate therapeutics are developed based on the trained model (operation 348). In one example embodiment, the model is used to find biomarkers for complex diseases from a vast set of data points. This accelerates therapeutic candidate discovery and enhances precision medicine efforts in healthcare where biomarkers relevant to an individual or a particular group of individuals can be observed using the multi-omics association learning method.

### Generate Associations Between Interpretable Multi-Omics Associations

Generally, CONMOAL can parse different multi-omics data, tokenize them and then learn relationships between the different features using the cross-modal attention between them. CONMOAL enables the handling of interactions between significant features from different modes.

### Many-to-Many Associations in CONMOAL

GWAS and other linear association methods generate single-marker associations for each genotype with respect to a phenotype or trait. Many-to-many associations are associations of multiple genotypes and multiple phenotypes found by one model, instead of learning multiple hypothesis tests. Multiple hypothesis testing often results in spurious associations which is avoided in many-to-many associations. Interpretability of biomarkers associated with complex diseases increases when looked at in the context of many variables considered in concert. These sometimes indicate underlying biological pathways and relationships between various biomarkers. In other words, interpretability increases with multiple genotypes linked with a group of phenotypes to indicate an underlying relationship between the two groups, possibly linked with biological functions of the genotypes and the subsequent response of a phenotype.

Cross-modal attention aggregates embeddings of two different data modalities and finds the attention weights attuned to both in a matrix. It enables the retrieval of one mode, e.g. IDP searching, by the other mode, e.g. SNPs. This generates a network of many-to-many associations and their relative importance. Conventional techniques use contrastive learning approaches in multi-omics data, but perform data integration for incomplete data and are, hence, suitable for a different use case. These techniques typically integrate incomplete data to attempt to form a complete picture of the multi-omics data and are less focused on the direct retrieval and many-to-many associations between distinct data types. This distinction makes cross-modal attention a powerful tool for specific applications where understanding the direct relationships and importance between different data types is critical.

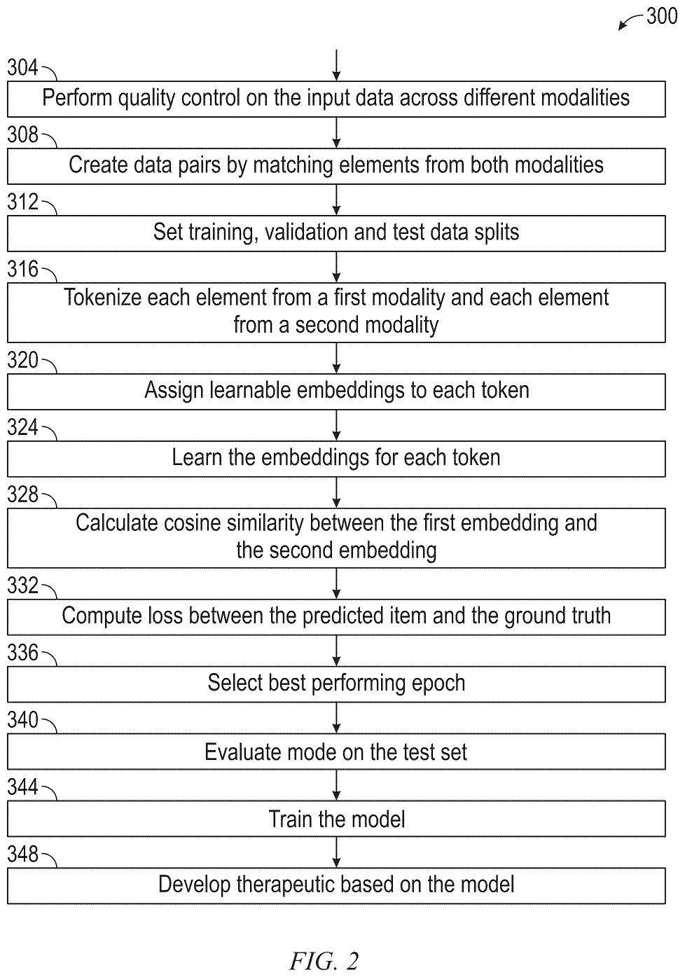

FIG. 3 is a table illustrating experimental results, in accordance with an example embodiment. CONMOAL obtained a 97.3% accuracy i.e., it was able to identify the SNP-IDP pairs with 97.3% accuracy on the test data set when trained using the top 0.5 percent SNPs (15 SNPs and 112 IDPs). The experimental results show the effective performance of CONMOAL to learn multi-omics associations. The SNP associations with complex disease from a conventional resource catalog were obtained for the experiments. The associations are denoted by the p-values as a marker for their strength. The top 1% of these associations were selected to consider for the pair making process. It was observed that the performance of CONMOAL decreases in response to increasing the percentage of associations that it is trained on. This happens as the top associations are the best representations of SNPs and IDPs as the SNPs are associated with neurological diseases which are in turn associated with the IDPs. As the number of associations considered are increased, more associations are allowed that exhibit decreasing strength and, hence, the decreasing strength also affects the quality of the representations learned.

The dataset includes 40,426 patients that have both modalities from a conventional data set. Experiments with the top 1% SNP selected result in a dataset of 33 SNPs and 112 IDPs. Positive pairs are obtained using diseases as proxy by matching each of the IDPs corresponding to a disease with each of the SNPs matching the same disease. The resulting dataset using the top 1% SNPs (in terms of the quality of the strongest relationships) has a training set size of approximately 27,000,000, a validation set of approximately 8,000,000, and a test set of approximately 4,000,000.

### Genome Tokenization

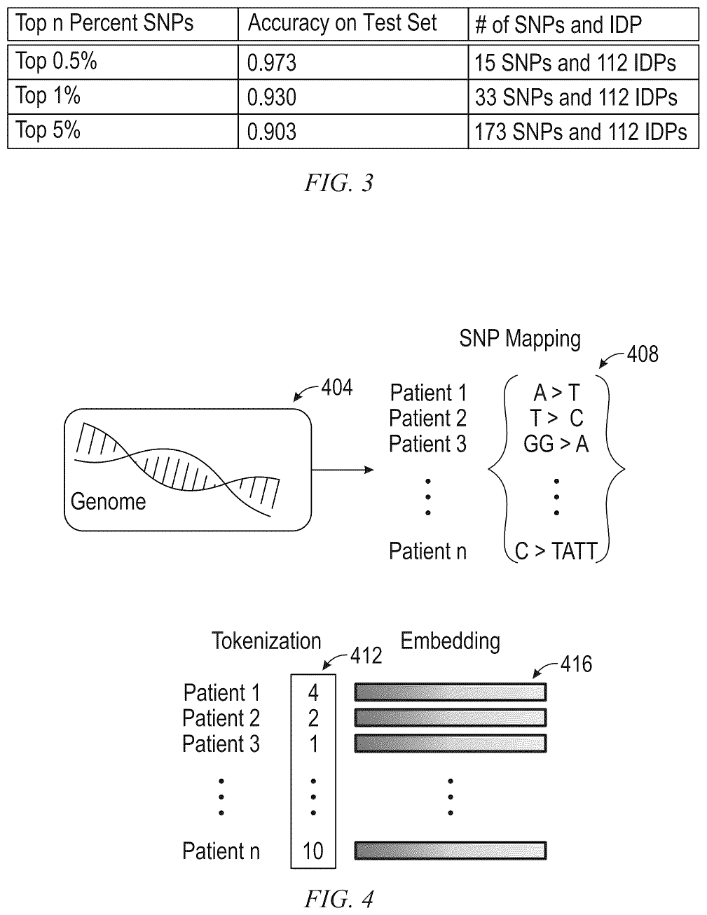

FIG. 4 illustrates tokenization of genome information for a given SNP, in accordance with an example embodiment. In one example embodiment, a genome 404 is analyzed to detect a list of mutations 408 for each patient. Each mutation 408 in each list is encoded (mapped) to a token 412 in a list of tokens 412 for each patient. The tokens 412 are used to generate learnable embeddings 416. Thus, the embeddings 416 capture the mutations 408 of each patient.

### Image Tokenization

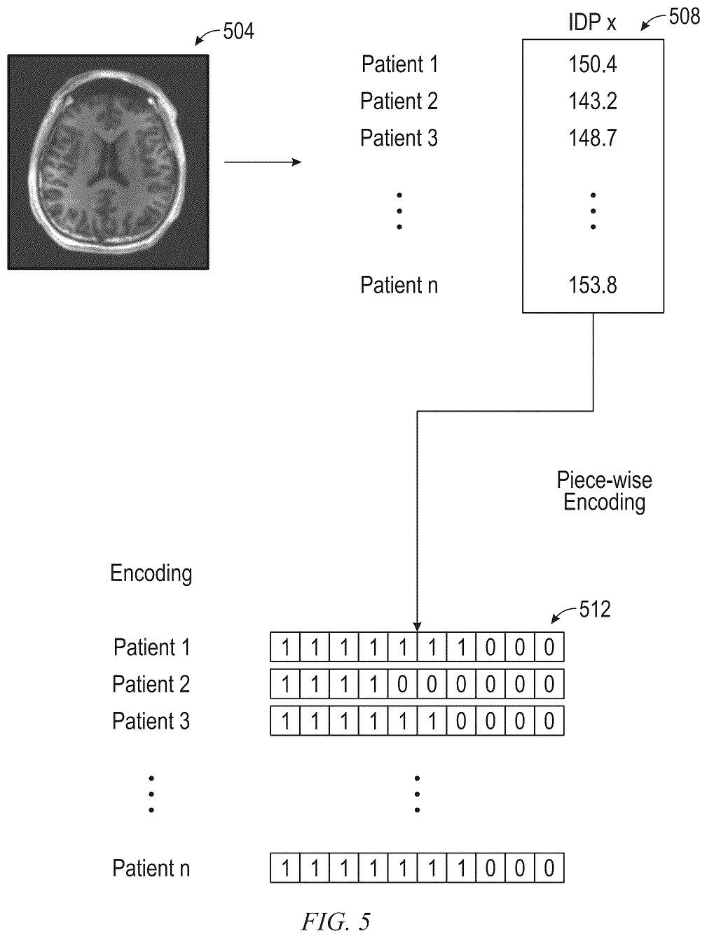

FIG. 5 illustrates tokenization of brain image information for a given imaging derived phenotype, in accordance with an example embodiment. In one example embodiment, a brain image 504 is analyzed to determine an IDP 508 for each patient. Each IDP 508 is piece-wise encoded into bins based on a distribution of the values of the encoded IDP. As described above, each IDP 508 is first binarized and embeddings are calculated for each sample based on their value relative to the rest of the samples. An n-dimensional vector 512 is built in which all the values are ones (1) before the main bin, and all the values following the main bin are zeros (0). The n-dimensional vectors 512 are used to generate learnable embeddings 416.

### Pair Making

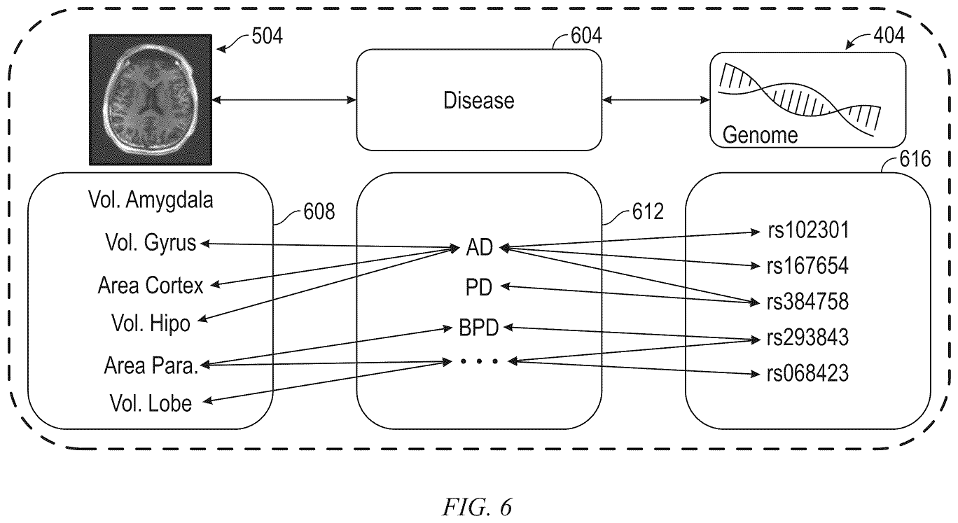

FIG. 6 illustrates an example of pair-making between IDPs 608 and SNPs 616, in accordance with an example embodiment. The pairs are based on a relationship between a brain image 504 and a genome 404 for a disease 604. Each disease of a set of diseases 612 is associated with an area of the brain 608 identified in the brain image 504 and one or more mutations 616 in the genome 404. As illustrated in FIG. 6, pairs have been filtered to those implicated by a known disease 612. In one example embodiment, the pair-making is performed using a canonical correlation analysis.

### Hyperparameters (Top 1% SNPs)

During experiments, the parameters below were observed to provide the best performance:

Learning rate: 0.00001

Best epoch: 3

Batch size: 7500

Model dimensions: 54

Number of transformer layers: 2

Dimensions of feed forward: 32

Dimensions MLP: 16

In one example embodiment, the GWAS catalog associations between SNPs and neurological conditions were used to select the SNPs for training data. Use of the whole genome sequencing and training on the entire sequence, with more compute resources, is contemplated to better represent the IDPs.

In addition, standard parameters recommended in the contrastive learning framework contrastive language-image pre-training (CLIP) were used. It is expected that hyperparameter tuning would provide more accuracy than observed. Similarly, including other data sources and increasing the number of IDPs used in training are expected to improve the representations and to increase accuracy.

As used herein, “facilitating” an action includes performing the action, making the action easier, helping to carry the action out, or causing the action to be performed. Thus, by way of example and not limitation, instructions executing on a processor might facilitate an action carried out by instructions executing on a remote processor, by sending appropriate data or commands to cause or aid the action to be performed. Where an actor facilitates an action by other than performing the action, the action is nevertheless performed by some entity or combination of entities.

Techniques as disclosed herein can provide substantial beneficial technical effects. Some embodiments may not have these potential advantages and these potential advantages are not necessarily required of all embodiments. By way of example only and without limitation, one or more embodiments may provide one or more of:
a contrastive multi-omics association learning (referred to by the short-hand form “CONMOAL” herein—references to CONMOAL should be understood as references to one or more exemplary embodiments thereof) platform capable of discovering many-to-many associations between any two modalities of multi-omics data;
a contrastive multi-omics association learning platform that can be fine-tuned to any multi-omics data set, even data sets having missing data;
accelerated discovery of multi-omics relationships through fine-tuning on genomic datasets and large biobanks;
a pretrained model that can be used as a reference for other methods to compare against or as a pretrained model for fine-tuning;
a contrastive multi-omics association learning platform that is disease agnostic as well as modality agnostic, generalizable to any health care data related to complex diseases;
a contrastive multi-omics association learning platform that enables biologically-meaningful and interpretable associations between pairs of multi-omics features to be discovered;
a disease-agnostic multi-omics model with self-supervised many-to-many association learning;
a disease-agnostic multi-omics model with cross-modal attention;
a pre-trained model for many-to-many multi-omics association discovery;
a contrastive multi-omics association learning platform that can be used in any setting that uses multi-modal features supported by CONMOAL;
a contrastive multi-omics association learning platform suitable for personalized medicinal efforts for use by industry, clinicians, researchers and the like;
a transfer-based multi-omics method to integrate various modalities of healthcare data including electronic medical records (EMR), imaging and multi-omics data, such as genomics, transcriptomics, and proteomics;
a non-linear way of computing high-dimensional multi-omics association studies (where high dimensions is on the order of hundreds of thousands to millions for mutations, 20,000-30,000 for genes and the like);
a method to learn many-to-many semantic relationships from different modalities useful for accelerating the discovery of therapeutics;
acceleration of the discovery of candidate therapeutics;
improvements to the technological process of computerized modeling and generation of therapeutics capable of identifying therapeutics for persons with genetic patterns that are not well represented in conventional data;
development of therapeutics for demographic groups not well represented in medical data repositories;
repurposing an existing drug as a therapeutic for another disease; and
a structure to find associations between different modalities of healthcare data and efficiently find the associated biomarkers for complex diseases, accelerating patent treatment and leading to the discovery of new therapeutic targets by considering the holistic effect of multiple modalities linked with the disease.

Given the discussion thus far, it will be appreciated that, in general terms, an exemplary method, according to an aspect of the invention, includes the operations of creating a plurality of data pairs by matching an element from a first modality with an element from a second modality (operation 308); tokenizing each element from the first modality and each element from the second modality to obtain first modality tokens and second modality tokens (operation 316); training a model based on the plurality of data pairs, the training comprising learning a first embedding from the first modality tokens via a first attention-based encoder 224 for the first modality and a second embedding from the second modality tokens via a second attention-based encoder 232 for the second modality (operation 324), calculating a cosine similarity between the first embedding 228 and the second embedding 236 for each data pair (operation 328) and computing a loss between predicted items and ground truth based on the cosine similarity (operation 332); and validating the predicted items with a minimal loss to obtain at least one candidate therapeutic.

In example embodiments, the validating is performed using biological pathway analysis and ontological studies. The information in DNA mutation is transferred through transcription to messenger RNA (mRNA), which in turn gets translated into proteins, which are the targets. Validation of this process is done via pathway analysis.

In example embodiments, a candidate therapeutic is developed based on the cosine similarity and the computed loss (operation 348).

In example embodiments, a patient is treated using the developed candidate therapeutic.

In example embodiments, an evaluation is carried out, the evaluation comprising selecting a best performing model and corresponding set of parameters where the best performing model is tested using a validation data set (operation 336); and evaluating a mode on a test data set by calculating accuracy, the evaluating further comprising determining, based on a given element of the first modality, whether the model retrieves a matching element of the second modality from the data pairs (operation 340).

In example embodiments, the trained model is fine-tuned on another training data set (operation 344).

In example embodiments, quality control is performed on each element from the first modality and each element from the second modality across different modalities by removing one or more individuals in the database 212 that are related to each other and removing individuals having relevant missing information from the database 212 (operation 304).

In example embodiments, a plurality of buckets of single nucleotide polymorphisms related to each of a plurality of diseases are established and a plurality of buckets of imaging-derived phenotypes related to each of the plurality of diseases are established; the data is expanded so that each data item becomes a combined record of SNP and imaging-derived phenotypes; and, for each disease, data dictionaries are created relating, for each imaging-derived phenotype, the single nucleotide polymorphism that is associated with a same disease (operation 308).

In example embodiments, the tokenizing of each element from the first modality comprises tokenizing single nucleotide polymorphisms by creating a dictionary of mutation types comprising a vocabulary of tokens; recoding, for each patient, a corresponding mutation status into one of the single nucleotide polymorphisms tokens; and selecting, using the tokenized representation, a corresponding learnable embedding (operation 316).

In example embodiments, the tokenizing each element from the first modality comprises analyzing a genome to detect a list of mutations for each patient and encoding each mutation in each list of mutations to a token in a list of tokens for each patient.

In example embodiments, a brain image is analyzed to determine an imaging-derived phenotype for each patient, each imaging-derived phenotype is piece-wise encoded into bins based on a distribution of values and an n-dimensional vector in which all values of the n-dimensional vector are ones before a main bin and all values of the n-dimensional vector are zeros following the main bin are generated, wherein the n-dimensional vectors are used to generate the embeddings.

In example embodiments, the learning the first embedding is based on a single nucleotide polymorphism and the learning the second embedding is based on image-derived phenotype.

In example embodiments, a matrix of the data pairs is generated by matching the first embedding and the second embedding, each cosine similarity is compared to a given threshold and the data pairs having a cosine similarity that exceeds the given threshold are kept.

In example embodiments, the computing the loss between the predicted items and the ground truth further comprises computing the loss using a first cross-entropy loss for the first modality and using a second cross-entropy loss for the second modality and averaging the first cross-entropy loss and the second cross-entropy loss.

In example embodiments, the tokenizing further comprises creating, for sequence data, a vocabulary based on all possible distinct elements; and employing, for tabular data, piecewise encodings.

In example embodiments, the first attention-based encoder and the second attention-based encoder are single-modality attention-based encoders.

In one aspect, a computer program product comprises one or more tangible computer-readable storage media and program instructions stored on at least one of the one or more tangible computer-readable storage media, the program instructions executable by a processor, the program instructions comprising creating a plurality of data pairs by matching an element from a first modality with an element from a second modality (operation 308): tokenizing each element from the first modality and each element from the second modality to obtain first modality tokens and second modality tokens (operation 316); training a model based on the plurality of data pairs, the training comprising learning a first embedding from the first modality tokens via a first attention-based encoder 224 for the first modality and a second embedding from the second modality tokens via a second attention-based encoder 232 for the second modality (operation 324), calculating a cosine similarity between the first embedding 228 and the second embedding 236 for each data pair (operation 328) and computing a loss between predicted items and ground truth based on the cosine similarity (operation 332); and validating the predicted items with a minimal loss to obtain at least one candidate therapeutic.

In one aspect, a system comprises a memory and at least one processor, coupled to the memory, and operative to perform operations comprising creating a plurality of data pairs by matching an element from a first modality with an element from a second modality (operation 308); tokenizing each element from the first modality and each element from the second modality to obtain first modality tokens and second modality tokens (operation 316); training a model based on the plurality of data pairs, the training comprising learning a first embedding from the first modality tokens via a first attention-based encoder 224 for the first modality and a second embedding from the second modality tokens via a second attention-based encoder 232 for the second modality (operation 324), calculating a cosine similarity between the first embedding 228 and the second embedding 236 for each data pair (operation 328) and computing a loss between predicted items and ground truth based on the cosine similarity (operation 332); and validating the predicted items with a minimal loss to obtain at least one candidate therapeutic.

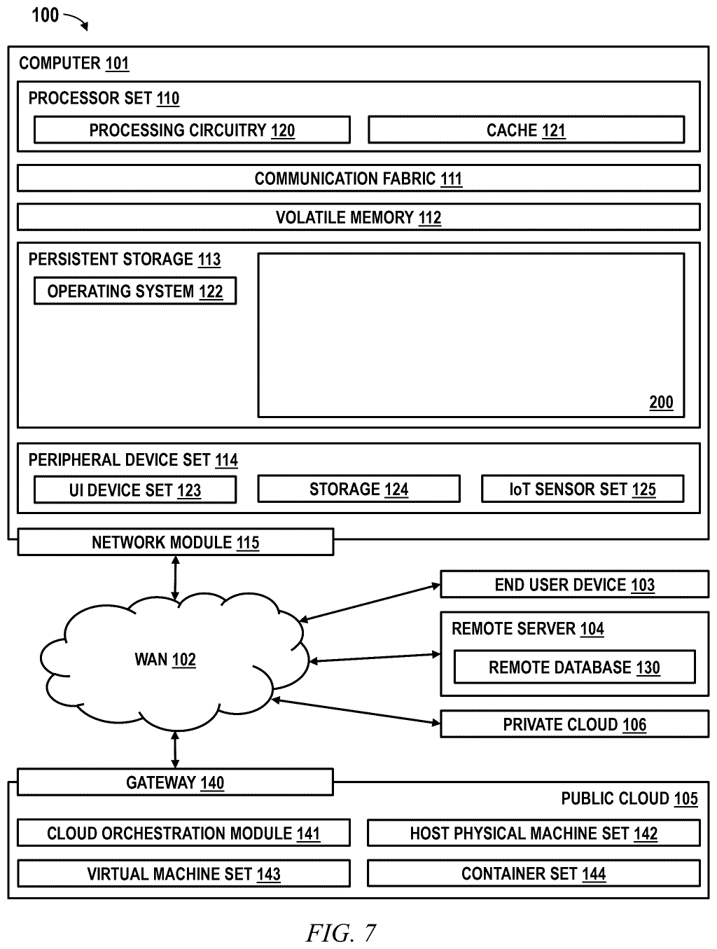

Refer now to FIG. 7.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as contrastive learning system 200 incorporating aspects of the invention. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 7. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economics of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method, comprising:
creating a plurality of data pairs by matching an element from a first modality with an element from a second modality;
tokenizing each element from the first modality and each element from the second modality to obtain first modality tokens and second modality tokens;
training a model based on the plurality of data pairs, the training comprising:
learning a first embedding from the first modality tokens via a first attention-based encoder for the first modality and a second embedding from the second modality tokens via a second attention-based encoder for the second modality;
calculating a cosine similarity between the first embedding and the second embedding for each data pair; and
computing a loss between predicted items and ground truth based on the cosine similarity; and
validating the predicted items with a minimal loss to obtain at least one candidate therapeutic.

2. The method of claim 1, wherein the validating is performed using biological pathway analysis and ontological studies.

3. The method of claim 1, further comprising developing the at least one candidate therapeutic based on the cosine similarity and the computed loss.

4. The method of claim 3, further comprising treating a patient using the developed candidate therapeutic.

5. The method of claim 1, further comprising:
carrying out an evaluation, the evaluation comprising:
selecting a best performing model and corresponding set of parameters where the best performing model is tested using a validation data set; and
evaluating a mode on a test data set by calculating accuracy, the evaluating further comprising determining, based on a given element of the first modality, whether the model retrieves a matching element of the second modality from the data pairs.

6. The method of claim 1, further comprising fine-tuning the trained model on another training data set.

7. The method of claim 1, further comprising performing quality control on each element from the first modality and each element from the second modality across different modalities by removing one or more individuals in the database that are related to each other and removing individuals having relevant missing information from the database.

8. The method of claim 1, further comprising:
establishing a plurality of buckets of single nucleotide polymorphisms related to each of a plurality of diseases and establishing a plurality of buckets of imaging-derived phenotypes related to each of the plurality of diseases;
expanding the data so that each data item becomes a combined record of SNP and imaging-derived phenotypes; and
creating, for each disease, data dictionaries relating, for each imaging-derived phenotype, the single nucleotide polymorphism that is associated with a same disease.

9. The method of claim 1, wherein the tokenizing of each element from the first modality comprises:
tokenizing single nucleotide polymorphisms by creating a dictionary of mutation types comprising a vocabulary of tokens;
recoding, for each patient, a corresponding mutation status into one of the single nucleotide polymorphisms tokens; and
selecting, using the tokenized representation, a corresponding learnable embedding.

10. The method of claim 1, wherein the tokenizing each element from the first modality comprises analyzing a genome to detect a list of mutations for each patient and encoding each mutation in each list of mutations to a token in a list of tokens for each patient.

11. The method of claim 1, further comprising analyzing a brain image to determine an imaging-derived phenotype for each patient, piece-wise encoding each imaging-derived phenotype into bins based on a distribution of values and generating an n-dimensional vector in which all values of the n-dimensional vector are ones before a main bin and all values of the n-dimensional vector are zeros following the main bin, wherein the n-dimensional vectors are used to generate the embeddings.

12. The method of claim 1, wherein the learning the first embedding is based on a single nucleotide polymorphism and the learning the second embedding is based on image-derived phenotype.

13. The method of claim 1, further comprising generating a matrix of the data pairs by matching the first embedding and the second embedding, comparing each cosine similarity to a given threshold and keeping the data pairs having a cosine similarity that exceeds the given threshold.

14. The method of claim 1, wherein the computing the loss between the predicted items and the ground truth further comprises computing the loss using a first cross-entropy loss for the first modality and using a second cross-entropy loss for the second modality and averaging the first cross-entropy loss and the second cross-entropy loss.

15. The method of claim 1, wherein the tokenizing further comprises:
creating, for sequence data, a vocabulary based on all possible distinct elements; and
employing, for tabular data, piecewise encodings.

16. The method of claim 1, wherein the first attention-based encoder and the second attention-based encoder are single-modality attention-based encoders.

17. A computer program product, comprising:
one or more tangible computer-readable storage media and program instructions stored on at least one of the one or more tangible computer-readable storage media, the program instructions executable by a processor, the program instructions comprising:
creating a plurality of data pairs by matching an element from a first modality with an element from a second modality;
tokenizing each element from the first modality and each element from the second modality to obtain first modality tokens and second modality tokens;
training a model based on the plurality of data pairs, the training comprising:
learning a first embedding from the first modality tokens via a first attention-based encoder for the first modality and a second embedding from the second modality tokens via a second attention-based encoder for the second modality;
calculating a cosine similarity between the first embedding and the second embedding for each data pair; and
computing a loss between predicted items and ground truth based on the cosine similarity; and
validating the predicted items with a minimal loss to obtain at least one candidate therapeutic.

18. A system comprising:
a memory; and
at least one processor, coupled to said memory, and operative to perform operations comprising:
creating a plurality of data pairs by matching an element from a first modality with an element from a second modality;
tokenizing each element from the first modality and each element from the second modality to obtain first modality tokens and second modality tokens;
training a model based on the plurality of data pairs, the training comprising:
learning a first embedding from the first modality tokens via a first attention-based encoder for the first modality and a second embedding from the second modality tokens via a second attention-based encoder for the second modality;
calculating a cosine similarity between the first embedding and the second embedding for each data pair; and
computing a loss between predicted items and ground truth based on the cosine similarity; and
validating the predicted items with a minimal loss to obtain at least one candidate therapeutic.

19. The system of claim 18, the operations further comprising:
carrying out an evaluation, the evaluation comprising:
selecting a best performing model and corresponding set of parameters where the best performing model is tested using a validation data set; and
evaluating a mode on a test data set by calculating accuracy, the evaluating further comprising determining, based on a given element of the first modality, whether the model retrieves a matching element of the second modality from the data pairs.

20. The system of claim 18, the operations further comprising:
establishing a plurality of buckets of single nucleotide polymorphisms related to each of a plurality of diseases and establishing a plurality of buckets of imaging-derived phenotypes related to each of the plurality of diseases;
expanding the data so that each data item becomes a combined record of SNP and imaging- derived phenotypes; and
creating, for each disease, data dictionaries relating, for each imaging-derived phenotype, the single nucleotide polymorphism that is associated with a same disease.
